(19) United States
(12) Patent Application Publication
(10) Pub. No.: US 2025/0160372 A1
(43) Pub. Date: May 22, 2025

(54) PROFILING, MODELING AND MONITORING TEMPERATURE AND HEAT FLOW IN MEAT OR FOOD ITEMS IN A COOKING PROCESS

(71) Applicant: GARDA TECH LTD, Beer Ganim (IL)

(72) Inventors: Sagie TSADKA, Meishar (IL); Netanel FARKASH, Beer Ganim (IL); Jeki DABACH, Ramat Gan (IL); Ori LAVIE, Herzlia (IL)

(21) Appl. No.: 18/839,034

(22) PCT Filed: Feb. 16, 2023
(86) PCT No.: PCT/IL2023/050170

§ 371 Date: Aug. 16, 2024

(51) Int. Cl. A23L 5/10; A47J 36/32; G01K 3/10; G01K 13/00

(52) U.S. Cl. CPC A23L 5/10; A47J 36/32; G01K 3/10; G01K 13/00

(57) ABSTRACT

The invention pertains to system and method for profiling, modeling and monitoring temperature and heat flow in a meat piece or food item in a cooking by placing the item on a flat surface of a cooking device in a sufficiently visible way for monitoring with an optical head, collecting data with suitable sensors and mirrors, such as IR sensor, visible range sensor, high resolution Lidar sensor, laser or LED device with different color options that has a collimated beam, scanning with a scanning mechanism, measuring temperature of bottom and upper surfaces of the item, modeling the item with a 3D as horizontally oriented equithermal slices and perpendicularly non-equithermal relative each other, and calculating temperature of these slices, particularly T_{core} of a central slice.

### TECHNICAL FIELD

The invention pertains to system and method for controlled and measurable cooking of meat items. Particularly, the invention pertains to method and system for modeling, calculating and monitoring temperature in a meat item and issuing instructions for timing the cooking stages, such as flipping the item, for obtaining optimal cooking.

### BACKGROUND

Monitoring a cooking process is necessary for optimizing the cooking of food items to obtain best results, customizing the cooking to any particular food item and saving investment of energy resources. Several apparatuses and methods are offered in this field, but none of them fully analyzes and models the cooking process itself, which leads to non-repetitive, non-controlled results as well as reduction in consumption of energy.

It is, therefore, an object of the present invention to provide a method and apparatus for resolving the shortcomings of those in the prior art.

It is yet another object of the present invention to provide a method for profiling, modeling and monitoring temperature and heat flow in meat pieces or food times in general in a cooking process.

It is yet another object of the present invention to provide a continuous profiling, modeling and monitoring method for optimizing the cooking process with dynamic, of the cooking of meat pieces and food items.

It is yet another object of the present invention to provide such a method for minimizing energy consumption in the cooking process.

It is yet another object of the present invention to provide an apparatus for profiling, modeling and monitoring temperature and heat flow in meat pieces or food times in general in a cooking process.

This and other objects of the present invention will become apparent as the description proceeds.

### SUMMARY

In one aspect, the present invention pertains to method and system for controlled and calculated cooking of meat items such as cattle, fish and poultry meat and the like. In one embodiment, the invention also pertains to controlled, measurable and/or calculated cooking temperature of other relatively flat and homogeneous food items. Particularly, the invention pertains to a method for measuring the initial conditions that characterize a food item and cooking appliance before start of the cooking, and calculating the spatially differential temperature of the item as it changes in real time.

In one embodiment, such calculation is done with a mathematical model that models the meat or food item as a three dimensional, 3D, body sliced horizontally relative to the cooking surface according to the temperature gradient from bottom to top. A heat flow equation with a finite element method is used to calculate the spatial temperature of any defined slice of the food item and the differential change between neighbor slices. Hands and sensor free monitoring of the food item is essential for automatic cooking. Sensors such as thermometers plugged into the item or manual intervention with kitchenware should no longer be required to provide real-time information on the cooking of the item and for obtaining optimal results. Instead, the method of the present invention enables real-time decisions such as when to flip the food item on the hot surface without the need for human or sensor intervention. In turn, this allows building robotic means that carries out an automatic cooking process.

In still another embodiment, the model is dynamic, time dependent and continuously updated. The values of different parameters of the system surroundings are measured by the sensors of the apparatus and updated in real-time. Then the parameters values are fed to the model for recalculating the temperature profile of the food item, particularly the temperature at its core, T_{core}, and dynamically reevaluating its temporal cooking state. Such dynamic and continuous variable value updating, recalculation and reevaluation improve the temperature modeling of the food item at any given time, especially T_{core} and ΔT_{core}, which is the temporal change in the temperature of T_{core}, and eventually optimizes the cooking process and the cooking of the food item.

In still another embodiment, the system is configured to simultaneously scan, monitor and cook a plurality of pieces of meat and/or food items or a plurality of pieces of meat of different types, cattle, poultry, fish and the like. At the same time, the system is configured to model every piece or item separately from other pieces/items and independently set their individual cooking plans as they dynamically change in the cooking process.

### BRIEF DESCRIPTION OF THE DRAWINGS

FIG. 1 illustrates schematics of the optical head and beam for scanning a meat piece or food item on cooking surface.

FIG. 2 illustrates temperature mapping of a meat piece or food item on a cooking surface.

FIG. 3 illustrates schematics of temperature differential modeling of a meat piece or food item.

FIG. 4 illustrates division of different components of a meat piece or food item.

FIG. 5 shows a table of relative content of different meat pieces.

FIG. 6 illustrates schematics of the optical head and temperature analysis and profiling of scanned meat pieces or food items.

### DETAILED DESCRIPTION OF THE DRAWINGS

### Scheme

The following drawing describes the cooking scheme of the meat/food item on a flat hot surface:

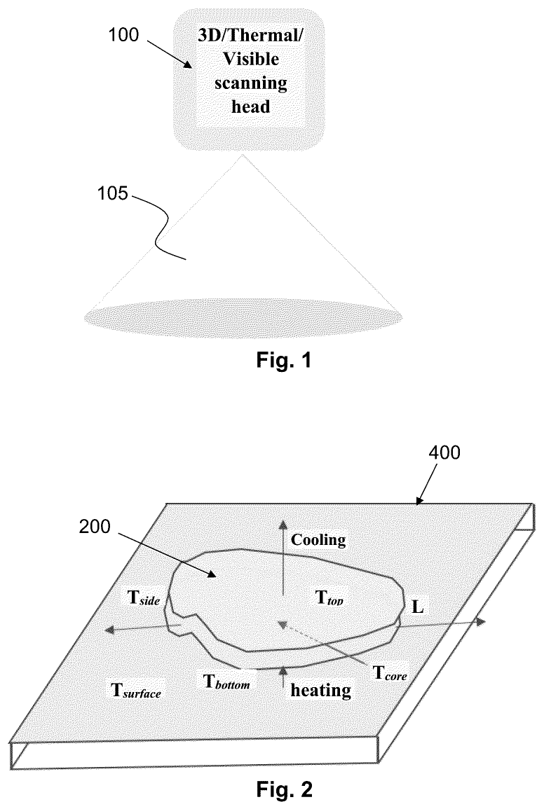

FIG. 1 schematically illustrates the system for measuring meat and surface temperatures in real time with a 3D, Thermal or Visible scanning head 100 that scans slice of meat (200, see FIGS. 2 and 4) with a beam 105, as well as the meat visible shape, size, texture of the meat and the surroundings. FIG. 2 illustrates the meat or food item 200, which are placed on a hot surface 400 with temperature T_{surface} and has initial temperature at all sides such that Tₜₒₚ=T_{bottom}=T_{side}, where T_{side} is the temperature at any side surface of the item 200; Tₜₒₚ is the temperature at the top exposed surface of the item 200; T_{bottom} is the temperature of the bottom surface of the item 200 facing the hot cooking surface.

An objective of the invention is to calculate the temporal ΔT_{core}, namely the differential of the temperature with time in a central slice as defined by the 3D model of the meat or food item 200 and horizontally oriented relative to the cooking surface 400. T_{core} is the temperature of a modeled central slice of the meat or food item 200, where the slice is horizontally equithermal with T_{core} at every point in the slice. On the other hand, the temperature of the item is perpendicularly non-equithermal, with a gradient of temperature from the bottom surface that interfaces the cooking surface 400 to the top exposed surface of the item 200.

T_{core} is calculated based on measurements done with an externally located 3D sensor that monitors the cooking apparatus and item 200 it cooks. ΔT_{core} is calculated relative to measured parameters of the cooking apparatus and surrounding conditions at any given change of temperature, ΔT, during cooking. An example of such surrounding conditions may be the temperature of the cooking surface. The non-equithermal and anisotropic characteristics of the meat or food item 200 in the perpendicular direction applies to the method of the invention for continuous monitoring and differential calculation of the temperature with time.

### Method

The method involves solving the heat flow equation of meat in real time. In first order, one can assume that a flat piece of meat is an isotropic 3D object. Heat flows into the meat bulk through the hot lower surface, and is conducted and released out through the exposed surfaces of the piece of meat or item, namely its upper and side surfaces. The first order approximation of the heat and temperature profile within the meat/item can be derived from solving a one dimensional heat flow equation, shown below, with a constant diffusion parameter of the meat/food:

$$\frac{\partial T}{\partial t} = D \frac{{\partial }^{2} T}{\partial {X}^{2}} ,$$

where—
X is the axis along which the temperature is measured,
T is the temperature of the measured item,
t is time, and
D is the diffusion constant.

The interpretation of this equation is that heat flows through the meat along the X coordinate at a rate that is proportional to the diffusion constant D and to the local second derivative of the temperature along the X direction. In general, this partial differential equation cannot be solved analytically, for most real life boundary conditions. We, therefore, solve it numerically using standard known methods for finite element calculations. Such methods may be the Crank-Nicolson method, forward and backward Euler and/or other known methods, all relevant to the method described here. Common to all these methods is the need to build a grid in space and time, where the calculation for the X axis is done in dx and dt steps, where dx and dt are small relative to the size of the object and dx=L/N where L is the height of the object and N the number of steps to calculate along the X axis. The Crank-Nicolson method, given that dt and dx are the calculation steps, can be represented in the following equation:

$$\frac{{T}_{i}^{n + 1} - {T}_{i}^{n}}{d t} = \frac{D}{2 d {x}^{2}} ( {T}_{i + 1}^{n} - 2 {T}_{i}^{n} + {T}_{i - 1}^{n} + {T}_{i + 1}^{n + 1} - 2 {T}_{i}^{n + 1} + {T}_{i - 1}^{n + 1} )$$

Where i represents the number of slices, each having a thickness of dx, and n represents the nₜₕ time step of the calculation. After introducing a new constant:

$$σ = \frac{D d t}{2 d {x}^{2}}$$

One can rearrange the equation in the following way:

$$( 1 + 2 σ ) {T}_{i}^{n + 1} - σ {T}_{i + 1}^{n + 1} - σ {T}_{i - 1}^{n + 1} = ( 1 - 2 σ ) {T}_{i}^{n} + σ 2 {T}_{i + 1}^{n} + σ {T}_{i - 1}^{n}$$

In this representation, the left side of the equation contains components of the temperature in various locations in the meat or food item but in a single time step n+1. The right side of the equation is a combination of temperatures in the time step n. Since we assume certain boundary and initial conditions at n=0 and n=N, the right side of the equation is known and therefore the left side can be developed to a set of equations that can be solved for any n for all the x values from i=0 to i=N.

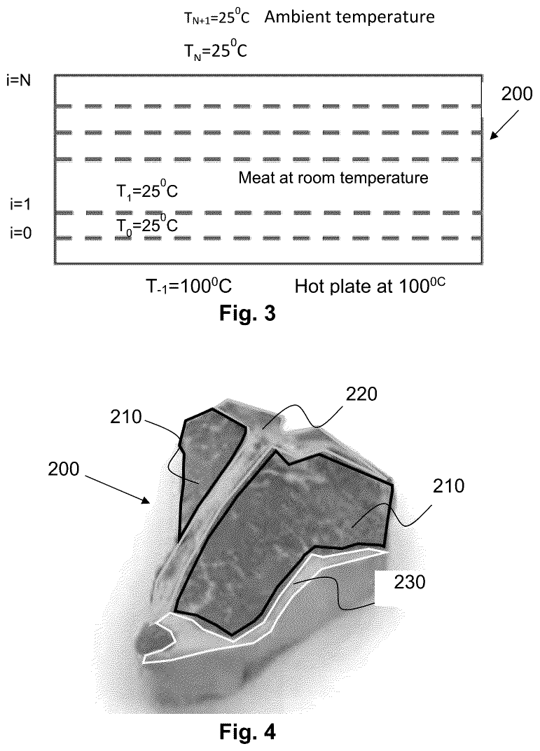

FIG. 3 describes the initial conditions that allow solving the set of equations above: In the time step n=0 the boundary conditions shown in FIG. 3 apply for the meat 200 on a hot plate.

The meat/item is placed on a hot surface with a known temperature of 100° C. in this case. The temperature in the modeled inner slices is in equilibrium with the ambient room temperature of 25° C. in this case. Therefore, all the Tis of the right side of the equation at n=0 are known and can be replaced with a constant vector, Aᵢ. On the left side of the equation, all the Th+1 values can be replaced with a new representation for simplicity, Uᵢ. Now the new set of equations will look like this:

$$i . ( 1 + 2 σ ) {U}_{i} - σ {U}_{i + 1} - σ {U}_{i - 1} = {A}_{i}$$

Developing this set of equations and placing number value to the index i we get:

$$( 1 + 2 σ ) {U}_{0} - σ {U}_{1} - σ {U}_{- 1} = {A}_{0} \qquad ( 1 )$$

$$( 1 + 2 σ ) {U}_{1} - σ {U}_{2} - σ {U}_{0} = {A}_{1} \qquad ( 2 )$$

$$( 1 + 2 σ ) {U}_{2} - σ {U}_{3} - σ {U}_{1} = {A}_{2} \qquad ( 3 )$$

$$( 1 + 2 σ ) {U}_{3} - σ {U}_{4} - σ {U}_{2} = {A}_{3} \qquad ( 4 )$$

$$⋮$$

$$∶$$

$$( 1 + 2 σ ) {U}_{N - 1} - σ {U}_{N} - σ {U}_{N - 2} = {A}_{N - 1} \qquad ( 5 )$$

In the first equation, U₋₁ is the temperature of the hot surface at the bottom that is measured by the sensor head that continuously, remotely monitors the cooking process. Therefore, we can replace it with the measured value and move it to the right side of the first equation. We then get the following equation:

$$( 1 + 2 σ ) {U}_{0} - σ {U}_{1} = σ {U}_{- 1} + {A}_{0} = {C}_{0} \qquad ( 6 )$$

Where Co is a known value. We can, therefore, express U₁ only as a function of U₀:

$${U}_{1} = \frac{( 1 + 2 σ ) {U}_{0} - {C}_{0}}{σ} \qquad ( 7 )$$

We can then introduce this U₁ expression into the second equation instead of the U₁ on its left side and get a second equation as follows:

$$( 1 + 2 σ ) \frac{( 1 + 2 σ ) {U}_{0} - {C}_{0}}{σ} - σ {U}_{2} - σ {U}_{0} = {A}_{1} \qquad ( 8 )$$

We can now express U₂ as a function of U₀ and introduce the expression of U₁ and U₂ into the third equation. Again we get an equation that involves only U₃ and U₀.

Repeating this process of replacement along the whole group and down to the last equation, we end up with the last equation that contains only expressions of U₀ and U_{N}. We now remember that U_{N}=T_{N}ⁿ⁺¹, which represents the temperature of the upper surface of the meat/item at a given time n+1, as remotely measured by the sensor head above the cooking plate. This value is measured in real time by the sensor head and can give the correct value for time n+1 for any n. Introducing this value into the last equation enables to calculate and solve U₀.

After solving for U₀, one can go back to the first equation, introduce the calculated value of U₀ and calculate U₁. Similarly, all the Uᵢs from U₁ to U_{N-1} are calculated in an iterative way. This basically gives us an accurate temperature profile of the meat/item in a time step n=1, from its lower surface up to its upper surface. This profile is a combination of heat flow calculations for the internal part of the meat/food item and measurements of the surface temperatures and the height profile of the meat, taken by the sensor head in real time.

We can now repeat this process to calculate the temperatures at n=2, n=3 etc. after substituting the results of the temperature profile for n=1, n=2 in the right side of the equation. This iterative process sets a method for predicting the internal temperature of the meat/food item while cooking on hot surface and monitoring with a sensor head as described above. This method is indifferent to exchanging positions between the top and bottom surfaces of the meat or food item, particularly flipping the meat if and when done in the cooking process. This is because the sensor head measures the top and bottom temperatures of the meat/item, and their values are introduced into the model in realtime. Therefore, flipping will not change the model progress and result. Rather the model will give a good fit to the core temperature with or without flipping throughout the process.

Based on its measurement and calculation capabilities, the system of the present invention is configured to re-evaluate calculated temperature values according to measured values, thereby more accurately predicting the core temperature value of the meat or food item. Thus, for example, when flipping a piece of meat, the system is configured to measure the actual temperature of the current top surface, compare it to its previous one which is used to calculate T_{core} and introduce the measured value into the set of equations to obtain a more accurate calculated core temperature value.

### The Diffusion Constant

D, the diffusion constant in the heat flow equation, is composed of the following parameters:

$$D = \frac{κ}{{pc}_{p}} ,$$

where—
K is the thermal conductivity of the meat/item,
p is the density, and
cₚ is the specific heat capacity of the meat.
D is measured in units of mm²/sec.

The density and specific heat capacity are distinguished for the different components that may be part of a slice of meat. Such components may include bones, fat, water and proteins. Typically, D will be selected to represent water that takes 70% on average of the mass of any meat that is cooked. D for lean meat, namely with low fat content, would be in the area of 0.12-0.14 mm²/sec. Using this value provides accurate temperature calculations. Based on https://www.tandfonline.com/doi/pdf/10.1271/bbb.58.1942, meat fat has lower diffusion value around 0.1 mm²/sec. Therefore, it is important to try and estimate the percentage of fat in the cooked meat to improve the accuracy of the model. Further, bones in the meat should be excluded from calculation, since they behave differently from meat and do not contribute for attributing a proper core temperature to the meat slice. Based on Heldman, https://meatscience.org/docs/default-source/publications-resources/rmc/1975/heat-transfer-in-meat.pdf?sfvrsn=e308bbb3_2, in the reference mentioned above, a 10% difference in thermal conductivity may also exist between directions parallel or perpendicular to the fibers of the meat, so cutting the meat may be important to set the proper D value.

It is, therefore, suggested in the method of the present invention to use the visual and thermal sensors of the system, which are part of the sensing head, to identify the areas of the meat before cooking, which are not to be controlled or measured in the process. Focusing on meat only areas will give better results of the model in predicting the core temperature of the meat. FIG. 4 is an example of a visual sensor looking on a piece of meat 200 from above and detecting the areas of the bone 220, fat 230 and meat 210. In this case, the model for controlled cooking will run only on the areas surrounded by the darker line that surrounds meat only areas 210. From a visual image, one can also detect the type of cutting of the meat (parallel or perpendicular to the fibers) and set a proper diffusion constant based on the type of cut.

A visible region image of the meat from the sensor head-detecting bone area (lighter line) 220, pure fat area (white line) 230 and meat area (black line) 210. In this case, the cooking model will run only on the area surrounded by the black line and will result with the most accurate core temperature estimation.

### Measuring Emissivity and Real Temperature

The emissivity of the surface of a material is its effectiveness in emitting energy as thermal radiation. Quantitatively, emissivity is the ratio of the thermal radiation from a surface to the radiation from an ideal black surface at the same temperature as given by the Stefan-Boltzmann law. The ratio varies from 0 to 1. Most organic materials have relatively high emissivity, but this parameter is no way constant and changes with the surface temperature, content and shape of the surface, of the monitored object. In order to accurately measure the temperature of a surface of an object one has to be able to estimate or measure its emissivity. The relation between temperature, emissivity and emitted thermal power is given by the Stefan-Boltzmann equation:

$$P = E A σ ( {T}^{4} - {T}_{c}^{4} )$$

where—
P is the emitted power in Watt,
A is the emitting area in m²,
ε is the emissivity, o is the Stefan-Boltzmann constant (5.67×10⁻⁸ Wm⁻²K⁻⁴),
T is the surface temperature in Kelvin, and
Tc is the ambient temperature in Kelvin.

The thermal sensor that is part of the scanning optical head that measures the meat from above, is measuring the parameter P/A (the emitted power per unit area of the meat, displayed on each pixel of the thermal sensor). For a known emissivity of the meat one can, therefore, use the equation above to calculate the accurate temperature of the upper surface of the meat. The ambient temperature may be measured by a simple thermometer or scanning optical head at certain pre-defined locations that are in equilibrium with the environment.

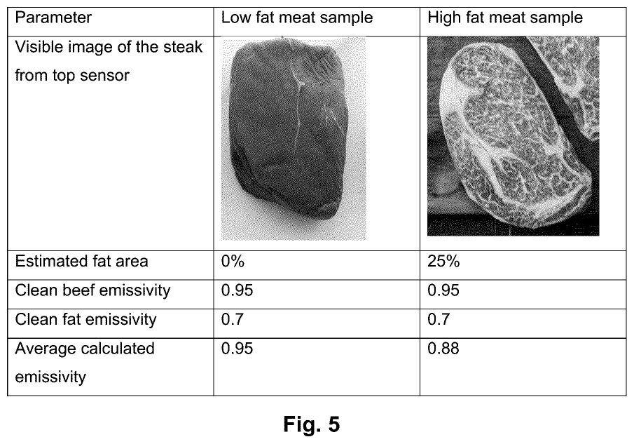

There are several methods that are suggested to estimate or measure the emissivity:
1. Estimate emissivity based on the visible structure of the meat: by analyzing the image taken by the visible sensor of the scanning optical head, one can decide how much does lean meat take part in the entire slice, and how much fat and other parts included in the upper surface of the meat. This analysis can be done with machine learning tools (such as automatic clustering and classification tools for visible images existing in the OpenCV library) or general image processing tools available from GPU manufacturers such as Nvidia, or by calculating the area of the different parts of the meat and giving weights to each part emissivity in the overall emissivity of the meat. One example of this last method is given in FIG. 5.
2. Measure the emissivity of the surface right after the flip of the meat on a hot surface-when a piece of meat is flipped, the lower surface of the meat that has been in contact with the hot surface, has a well-known temperature that is equal to the hot surface temperature. This will stay constant over a very short time after flipping, and if in that short time frame the scanning optical head will grab a thermal image of the meat, one will have knowledge of the real meat temperature and will be able to accurately calculate the emissivity of that surface using the Stephan Boltzman equation above. One can then use this emissivity value for further temperature calculation over time when the upper surface changes its structure from fresh meat to burnt meat (with denaturized protein).
3. Estimate and measure both the temperature and emissivity of the meat based on the amount of water that releases off of the meat. The amount of water on top of the upper surface or lower hot surface is related directly to the proteins denaturation inside the meat and takes place at certain temperatures that are known in the professional literature. The optical head with its sensor in the visible range can detect water content in the image and estimate its quantity change over time by simple image processing tools. After estimating the percentage of water change one can estimate the temperature and more importantly, the emissivity of the meet at this stage, and update the heat flow model with updated emissivity numbers.
4. Emissivity values for various meat, fish and chicken pieces can be estimated better over time if one measures the real core temperature versus the calculated core temperature from the model described above. In this case, one can change the emissivity value that is used in the model to a value that will give equal core temperatures between model and measurement. This can be done in an iterative way and in small steps of emissivity values that are introduced into the model until we get same results of core temperature. Once we find the emissivity, we can store it in memory, together with information about the type of meat, temperature range, characteristics of the meat (fat, protein, water content, bones, color etc.) and any other important parameter of the cooking process. These measurements of corrected emissivity can be taken sporadically once every few cooking cycles or even with a single sample out of several tens or hundreds of cooking cycles. Once we accumulate enough emissivity data points in various meat types and cooking conditions, we can use standard data mining algorithms to run over the data base and recommend the right emissivity for future cooking cycles depending on the meat and tools to be used.

### The Scanning Optical Head

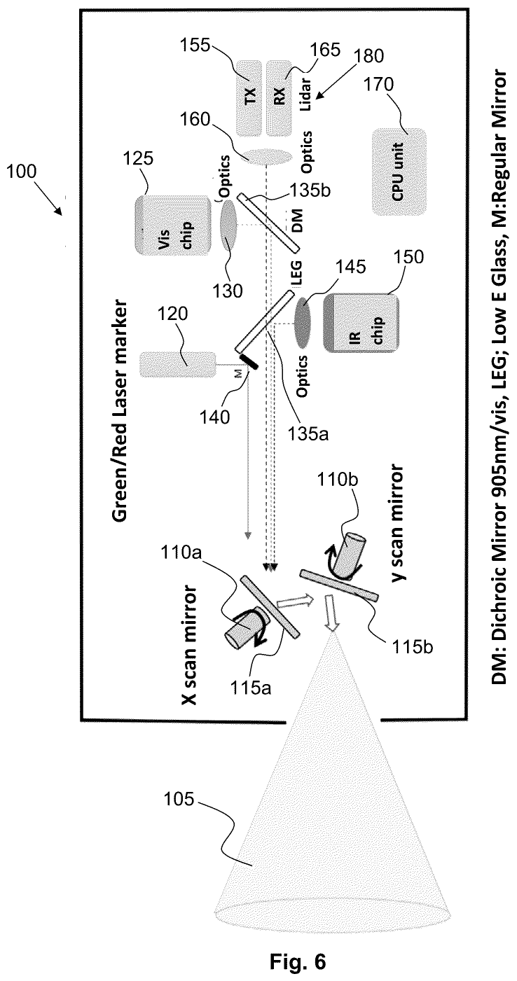

The scanning optical head 100 that is located above the cooking plate measures all the meat and cooking tool parameters that are important for solving the heat flow equation and heat flow model described above in real time. One suggested structure of such an optical head is given in FIG. 6. The design allows placing the optical head 100 at any distance and angle above the cooking plate, and the system performs well, provided it has a clear line of sight to the cooking plate and meat/item cooked. The system can automatically calibrate its area of interest for monitoring the cooking process by using depth, range and visible image data of the area that is monitored. The system 100 contains a processor 170 and memory and communication units that accumulate the data collected by the sensors in real time, run the heat flow model and solve it in real time, and combine various images from the various sensors into one unified database that helps making decisions regarding the cooking process that is monitored.

The scanning optical head contains the following components:
1. An IR (infrared) chip 150 that is sensitive to the thermal heat radiated from the food and hot plate, and other parts surrounding the cooking area. The chip 150 has its own unique optics 145 to set its field of view, focal range and resolution, and can include a variable focal point, zoom, aperture and any other optical parameter used in such type of sensors. Given the emissivity of the object, this IR chip 150 and optics 145 can measure the surface temperature of the object being observed with high accuracy, very high resolution and high frame rate of at least 25 Hz.
2. A visible range chip 125 with its own optics 130—that is sensitive to the visible range of the spectrum and can detect, monitor and measure objects with color information in real time at a rate of at least 60 Hz and HD resolution.
3. A high resolution Lidar sensor 180 combined from transmitter 155 and receiver 165 with its own optics 160 that can measure the range and depth of any object with depth resolution greater than 2 mm, and horizontal and vertical resolution with the same accuracy.
4. A laser or LED device 120 with different color options that has a collimated (collimator 140) beam and can illuminate an object on the cooking surface from a distance and mark the object with different colors and/or different flash rates to visually signal information about the status of the object or surface, e.g., hot, cold, above certain temperature, ready, remove from surface, flip etc.
5. A set of mirrors 135a, dichroic mirrors 135b and low emissivity glass windows that are aligned along the optical path of all the sensors described above and allow combining all the sensors line of sights into a single axis with no parallax between the sensors and with optimal efficiency. This set of mirrors and reflectors allows having one single aperture of the system, through which all the sensors operate, and the images that are created by all the sensors are fully aligned with no need for spatial calibration.
6. A two-dimensional scanner 110a, 110b with two scanning mirrors 115a, 115b, or other known scanning mechanism, that is positioned right before the main aperture of the optical head and can scan the optical axis of the system which allows for the various sensors included in the optical head to see various directions in space and cover large field of view while still keeping the high resolution of the sensor and the image it creates. The scanner can operate in various speeds and scan rates and can serve as a scanner for various functions. There are several functionalities for the scanner:
   a. Serve as an individual scanner for one of the sensors while the others are idled-scan the field of view of one sensor with its optimal scan rate and scan pattern where the other sensors are either shut down or blocked in order to avoid misreading of their image.
   b. Serve as a scanner for all the sensors together—where in this case the scanner scans the field of view for all the sensors that are aligned along a single optical axis, and in this mode the scan rate and scan pattern is optimized for all the sensors and not to one specific sensor.
   c. Calibration mode—the scan unit moves in small steps to detect the corners of the required scan area, or moves to certain points in space to allocate a reasonable area to be monitored by the optical head—for instance—allocate the corners of a grilling device and a hot plate and set this area as the system monitoring area for the cooking process.
   d. High resolution mode-scan a specific area with very small steps to accumulate multiple images of same area—and in this way get higher pixel resolution for a specific area on the cooking surface.
   e. Large field of view mode-Move the scanning unit in lower speed and accumulate multiple images of the various sensors to create a single combined image from every sensor. The combined image has a much larger cover area with resolution similar to that of the instantaneous image of each one of the sensors.
   f. Mark mode—The 2D scanner can direct the laser or LED light which is part of the optical head in a way that creates clear light markers on the cooking surface or on and around the food being cooked-laser beam manipulation by mirror scanning is well known in areas like laser shows, and similar techniques known in the literature can be used here to mark specific items and functions by light.
7. A CPU unit 170 is integrated into the optical head or connected to its components through a communication line. This unit can be based on, for example, an nVIDIA Jetson Nano GPU processor, that is specifically designed to run advanced AI and neural networks algorithms on large images of various types, and can connect to multiple cameras and sensors, acquire their images in real time and perform strong image processing and object classification in between frames. The CPU unit can control, communicate and send commands to multiple external sensors and devices, as required in the system described above. The following functions of the CPU are to be mentioned:
   a. The CPU unit connects to the optical head sensors, acquires the images in real time, and processes the images based on various algorithms.
   b. When running the heat transfer model described above, and by using the stored data from all the sensors, the CPU can monitor, calculate and maintain the internal core temperature of the food being cooked.
   c. By using AI algorithms, the CPU can verify the quality of cooking through the whole cooking process (by identifying temperature, color, shape, smoke level, size and shape changes and more).
   d. The CPU communicates with external monitors and/or database through a communication line—either physical or wireless.
   e. The CPU Collects and stores data for future use—including improving the learning process of the algorithms, monitoring the performance of kitchen teams while cooking dishes, and other use cases for the data.
   f. The CPU is capable of running strong AI algorithms, including such that are based on Neural Networks, to decide in real time on the cooking quality, and change the energy flow to the cooking tool to optimize the cooking process, and save energy consumption (gas, electricity etc.).
   g. The CPU can also send data to a server in the cloud, where such data can include, for example, images of dishes while being cooked, power consumption data, gas reserve values, and other relevant data points.
   h. The CPU can send alarms in various ways—sound, light, calling to a fixed line or mobile phone etc.—in order to alert on dangerous situations such as smoke, fire, hot parts in front of people using the stove, spilled liquids on surfaces, overcooking of food etc.
   i. The CPU can connect to other sensors such as temperature, smoke, CO and other dangerous gases, earthquake sensors etc. By connecting to these sensors, the CPU can give alerts on various situations that need the human intervention such as qualified personnel in the process or alert on dangerous situations.
   j. The CPU can connect to the user via a communication fixed line, ethernet line, Wifi, Bluethooth, NFC, RF, or other remote control wireless solutions that exist and are common in use at the time of implementation of the solution.

The capabilities and functionalities of the scanning optical head combined with the modeling and mathematical calculations in the CPU enable to obtain accurate cooking temperatures and a finely cooked piece of meat or any other food item. The scanning optical head provides realtime data on the cooked item, its parts and content, the boundaries of the meat/item part that is monitored, its surroundings and cooking appliance. These can be used to reevaluate the diffusion constant that depends on the thermal conductivity of the meat/item, its density and specific heat capacity. The new value of the diffusion constant can be fed back to the set of equations to obtain a more accurate value of T_{core}.

1. A method for profiling, modeling and monitoring temperature and heat flow in a meat piece or food item in a cooking process, said method comprising:
placing said meat piece or food item on a flat surface of a cooking device, where said meat piece or food item and flat surface are sufficiently visible for monitoring with an optical head that comprises suitable sensors, a set of mirrors that combine all the sensors line of sights into a single axis with no parallax between them and a two-dimensional scanner with a scanning mechanism;
measuring temperature of bottom surface of said meat piece or food item that interfaces the flat surface of the cooking device and temperature of upper surface of said meat piece or food item opposite the bottom surface and exposed to the surrounding of the cooking device;
modeling said meat piece or food item with a 3D model that dissects them to horizontally oriented equithermal slices and perpendicularly non-equithermal relative each other; and
calculating temperature of these slices, with a set of equations for making such calculations and based on the measured temperatures of the bottom and upper surfaces of said meat piece or food item.

2. The method according to claim 1, wherein said sensors comprise IR sensor, visible range sensor, high resolution Lidar sensor, laser or LED device with different color options that has a collimated beam.

3. The method according to claim 1, wherein said scanning mechanism comprises two scanning mirrors.

4. The method according to claim 1, wherein said calculating temperature of said slices comprises calculating T_{core} of a central slice in middle of the modeled meat piece or food item.

5. The method according to claim 1, further comprising ΔT_{core}, which is the temporal change in the temperature of T_{core} of the central slice in the cooking process and as modeled in the 3D model.

6. The method according to claim 5, wherein said ΔT_{core} is dynamically and continuously updated in said cooking process, wherein said 3D model is dynamically and continuously fed with parameter values of surrounding of said meat piece or food item for recalculating and reevaluating temperature modeling of said meat piece or food item and optimizing said cooking process and cooking of said meat piece or food item.

7. The method according to claim 6, wherein said parameters are selected from bottom and upper surface temperature of said meat piece or food item and thickness of said meat piece or food item.

8. The method according to claim 1, wherein the 3D model of the piece of meat or food item distinguishes between different components of meat piece or food item, and wherein said 3D model is constructed solely on lean meat.

9. The method according to claim 8, wherein said different components of said meat piece or food item comprise bones, fat, water and proteins.

10. The method according to claim 1, further comprising simultaneously scanning, monitoring and cooking a plurality of pieces of meat and/or food items or a plurality of meat pieces of different types and modeling every piece or item separately from other pieces/items and independently setting individual cooking plans dynamically changed in the cooking process.

11. The method according to claim 10, wherein said different pieces of said meat pieces comprise cattle, poultry and fish.

12. The method according to claim 1, further comprising reevaluating calculated temperature values according to measured values, more accurately predicting core temperature value of said meat piece or food item.

13. The method according to claim 8, further comprising flipping said meat piece or food item, measuring actual temperature of current top surface, comparing said actual temperature to previous actual temperature which is used to calculate T_{core} and introducing measured value of said actual temperature of said current top surface into said set of equations to obtain a more accurate calculated core temperature value.

14. The method according to claim 1, further comprising obtaining realtime data on the cooked item, its parts and content, the boundaries of the meat/item part that is monitored, its surroundings and cooking appliance with the scanning optical head combined with the modeling and mathematical calculations in the CPU, reevaluating the diffusion constant that depends on the thermal conductivity of the meat/item, its density and specific heat capacity and feeding it back to the set of equations to obtain a more accurate value of T_{core}, for more accurate cooking temperatures and a finely cooked meat piece or food item.

15. A system for profiling, modeling and monitoring temperature and heat flow in a meat piece or food item in a cooking process, said system comprising:
a cooking device with a flat surface that is suitable for cooking pieces of meat and food items;
an optical head that comprises suitable sensors, a set of mirrors that combine all the sensors line of sights into a single axis with no parallax between them, and a two-dimensional scanner with two scanning mirrors or other known scanning mechanism, where the optical head is configured to measure temperature of bottom and top surfaces of the meat piece or food item and their dimensions, shape, boundaries, height, width and 3D structure that are important to the accurate solution of a 3D heat flow model, taken in real time;
software means for 3D modeling, dissecting the meat piece or food item to slices horizontally relative to the bottom and top surface of the meat piece or food item and calculating temperature T_{core} of a defined slice at center of the meat piece or food item and ΔT_{core} of temporal change in temperature during cooking; and
a CPU for receiving data collected from the sensors and calculations and producing values of T_{core} and ΔT_{core}.

16. The system according to claim 15, wherein said suitable sensors are selected from IR sensor, visible range sensor, high resolution Lidar sensor, laser or LED device with different color options that has a collimated beam.
